(19) United States
(12) Patent Application Publication
(10) Pub. No.: US 2005/0154196 A1
(43) Pub. Date: Jul. 14, 2005

(54) Process for producing sugar in which molecular weight is lowered

(75) Inventors: Hiroshi Maeda, Yokosuka-shi (JP); Kimihiro Imamura, Yokosuka-shi (JP)

Correspondence Address: SUGHRUE MION, PLLC, 2100 PENNSYLVANIA AVENUE, N.W., SUITE 800, WASHINGTON, DC, 20037, US

(73) Assignee: SEIKAGAKU CORPORATION

(21) Appl. No.: 10/755,667

(22) Filed: Jan. 13, 2004

(51) Int. Cl. C07H 5/04; C07H 5/06; C08B 37/00; C08B 37/10

(52) U.S. Cl. 536/18.7; 536/21; 536/54; 536/55.2; 536/55.3; 536/123.1; 536/124; 106/162.2; 106/162.1

(57) ABSTRACT

A process for producing a saccharide having a lowered molecular weight, which comprises irradiating an electron beam to a polysaccharide fraction. Also, a process for producing a saccharide having a weight average molecular weight of n (Da), which comprises irradiating an electron beam to a polysaccharide fraction which has a weight average molecular weight of M (Da) and is in a solid state at a dosage of d (kGy) that satisfies the following equation:
n=Me^{ad} (wherein M is a number of 5,000 to 70,000; n is an optional positive number; e is the base of natural logarithm; and a is a number of −0.008 to −0.004). Furthermore, a process for producing hyaluronic acid having a lowered molecular weight, which comprises irradiating an electron beam to a hyaluronic acid fraction.

### BACKGROUND OF THE INVENTION

1. Field of the Invention

The present invention relates to a process for producing a saccharide having a lowered molecular weight and a method for lowering the molecular weight of a polysaccharide.

2. Brief Description of the Background Art

First, units, abbreviations and terms used herein are explained.
Da: Dalton
Gy: gray
kGy: kilogray
GAG: glycosaminoglycan
HA: hyaluronic acid
CS: chondroitin sulfate
CS-A: chondroitin sulfate A
CS-C: chondroitin sulfate C
CS-D: chondroitin sulfate D
CS-E: chondroitin sulfate E
DS: dermatan sulfate (also called chondroitin sulfate B)
KS: keratan sulfate
HS: heparan sulfate
Hep: heparin

The term “average molecular weight” as used herein means a weight average molecular weight.

Those which have various pharmacological activities and biological activities have been reported as saccharides having a lowered molecular weight. For example, it has been reported that a saccharide in which the molecular weight of HA is lowered has activity such as growth inhibition of a cancer cell, expression reinforcement of a heat shock protein, cell death inhibition, and cytotoxicity inhibition. Also, it has been reported that a saccharide in which the molecular weight of KS is lowered has activity such as anti-inflammatory, anti-allergic, and immune-modulating. In addition, it has been reported that a saccharide in which the molecular weight of Hep is lowered has activity such as affinity for a growth factor.

Thus, since a saccharide having a lowered molecular weight has a possibility as a raw material of medicaments, food and the like, it is preferred that it can be mass-produced conveniently at a low cost.

In the process for producing a saccharide having a lowered molecular weight, generally, a polysaccharide to be used as the production raw material is dissolved in a solvent, and its molecular weight is lowered by allowing acid, alkali, enzyme or the like to act upon the raw material. However, when a solid state polysaccharide is used as the production raw material, it is necessary to carry out the reaction after dissolving it in a solvent, and a certain treatment (neutralization, termination of enzyme reaction or the like) is required even after the reaction. The circumstances regarding labor and time for such a treatment and control, handling, cost and the like of a reagent to be added (acid, alkali, enzyme or the like) are considered, so that a method by which a polysaccharide having a lowered molecular weight can be mass-produced conveniently and quickly at a low cost has been desired.

### SUMMARY OF THE INVENTION

An object of the present invention is to provide a process which can mass-produce a polysaccharide having a lowered molecular weight conveniently and quickly at a low cost and a method which can lower the molecular weight of a polysaccharide in a large quantity.

In order to solve the above-described problems, the present inventors have conducted intensive studies and found as a result, (1) the molecular weight of a polysaccharide is lowered by irradiation of an electron beam, (2) the molecular weight is also lowered when an electron beam is irradiated to the polysaccharide in a solid state, (3) the irradiation of the electron beam does not have fatal influence upon the skeletal structure of the polysaccharide, (4) even in the case where the polysaccharide contains a sulfate group, influences of the electron beam irradiation such as loss of the sulfate group are hardly generated, (5) the polysaccharide having a molecular weight lowered to an almost constant average molecular weight can be surprisingly produced when a certain level of dosage of the electron beam is irradiated, almost independent of the average molecular weight of the polysaccharide to be used as the production raw material, and this tendency is significant particularly when the average molecular weight of the polysaccharide to be used as the production raw material is large (particularly HA), and (6) when the average molecular weight of the polysaccharide to be used as the production raw material is within a certain range, a certain relational equation is formed among the average molecular weight of the polysaccharide to be used as the production raw material, the dosage of the electron beam and the average molecular weight of the produced saccharide having a lowered molecular weight. Thus, a process which can mass-produce a polysaccharide having a lowered molecular weight conveniently and quickly at a low cost and a method which can lower the molecular weight of a polysaccharide in a large quantity can be provided, and thus the present invention has been accomplished.

### DETAILED DESCRIPTION OF THE INVENTION

Accordingly, the present invention provides a process for producing a saccharide having a lowered molecular weight, which comprises at least a step of irradiating an electron beam to a polysaccharide fraction (hereinafter referred to as “production process of the present invention”).

Also, as a preferred embodiment of the production process of the present invention, the present invention provides a process for producing a saccharide having an average molecular weight n (Da), which comprises at least a step of irradiating an electron beam to “a polysaccharide fraction which has an average molecular weight of M (Da) and is in a solid state” at a dosage of d (kGy) which satisfies the following equation:
n=Me^{ad}
(wherein M is a number of 5,000 to 70,000; n is an optional positive number; e is the base of natural logarithm, and a is a number of −0.008 to −0.004) (hereinafter referred to as “production process 1 of the present invention”).

The “a” is preferably a number of −0.008 to −0.005, more preferably a number of −0.0075 to −0.0050.

According to the production process of the present invention, the “polysaccharide fraction” to which the electron beam is irradiated is preferably a GAG fraction. The “GAG fraction” is preferably a fraction comprising at least one species of GAGs selected from the group consisting of HA, CS (including CS-A, CS-C, CS-D, CS-E and the like), DS, KS, HS and Hep.

The present invention also provides a method for lowering the molecular weight of a polysaccharide, which comprises irradiating an electron beam to a polysaccharide fraction (hereinafter referred to as “molecular weight-lowering method of the present invention”).

Also, as a preferred embodiment of the molecular weight-lowering method of the present invention, the present invention provides a method for lowering the molecular weight of a polysaccharide to an average molecular weight of n (Da), which comprises irradiating an electron beam to “a polysaccharide fraction which has an average molecular weight of M (Da) and is in a solid state” at a dosage of d (kGy) which satisfies the following equation:
n=Me^{ad}
(wherein M is a number of 5,000 to 70,000; n is an optional positive number, e is the base of natural logarithm; and a is a number of −0.008 to −0.004) (hereinafter referred to as “molecular weight-lowering method 1 of the present invention”).

The “a” is preferably a number of −0.008 to −0.005, more preferably a number of −0.0075 to −0.0050.

According to the molecular weight-lowering method of the present invention, the “polysaccharide fraction” to which the electron beam is irradiated is preferably a GAG fraction. The “GAG fraction” is preferably a fraction comprising at least one species of GAGs selected from the group consisting of HA, CS (including CS-A, CS-C, CS-D, CS-E and the like), DS, KS, HS and Hep.

Also, as a preferred embodiment of the production process of the present invention, the present invention provides a process for producing HA having a lowered molecular weight, which comprises at least a step of irradiating an electron beam to an HA fraction (hereinafter referred to as “production process 2 of the present invention”).

Also, as a preferred embodiment of the production process 2 of the present invention, the present invention provides a process for producing HA having a lowered molecular weight, which comprises at least a step of irradiating an electron beam to “an HA fraction which has an average molecular weight of 5,000 to 3,000,000 (Da) and is in a solid state” at a dosage of 5 to 400 (kGy). In addition, the following preferred embodiments of this production process are provided:
the production process wherein the “HA fraction” to which the electron beam is irradiated has an average molecular weight of 200,000 to 3,000,000 (Da); the dosage is from 5 to 15 (kGy); and the average molecular weight of the “HA having a lowered molecular weight” is from 100,000 to 200,000 (Da);
the production process wherein the “HA fraction” to which the electron beam is irradiated has an average molecular weight of 100,000 to 3,000,000 (Da); the dosage is from 15 to 30 (kGy); and the “HA having a lowered molecular weight” has an average molecular weight of 60,000 to 100,000 (Da);
the production process wherein the “HA fraction” to which the electron beam is irradiated has an average molecular weight of 50,000 to 3,000,000 (Da); the dosage is from 30 to 50 (kGy); and the “HA having a lowered molecular weight” has an average molecular weight of 30,000 to 60,000 (Da);
the production process wherein the “HA fraction” to which the electron beam is irradiated has an average molecular weight of 30,000 to 3,000,000 (Da); the dosage is from 50 to 150 (kGy); and the “HA having a lowered molecular weight” has an average molecular weight of 20,000 to 30,000 (Da);
the production process wherein the “HA fraction” to which the electron beam is irradiated has an average molecular weight of 20,000 to 3,000,000 (Da); the dosage is from 150 to 250 (kGy); and the “HA having a lowered molecular weight” has an average molecular weight of 10,000 to 20,000 (Da); and
the production process wherein the “HA fraction” to which the electron beam is irradiated has an average molecular weight of 5,000 to 3,000,000 (Da); the dosage is from 250 to 350 (kGy); and the “HA having a lowered molecular weight” has an average molecular weight of 3,000 to 10,000 (Da).

Also, as another preferred embodiment of the production process 2 of the present invention, the present invention provides a process for producing HA having a lowered molecular weight, which comprises at least a step of irradiating an electron beam “an HA fraction which has an average molecular weight of 600,000 to 1,200,000 (Da) and is in the liquid state” at a dosage of 10 to 80 (kGy) to. In addition, the following preferred embodiments of this production process are provided:
the production process wherein the “HA fraction” to which the electron beam is irradiated has an average molecular weight of 600,000 to 1,200,000 (Da); the dosage is from 10 to 30 (kGy); and average molecular weight of the “HA having a lowered molecular weight” is from 2,500 to 4,000 (Da);
the production process wherein the “HA fraction” to which the electron beam is irradiated has an average molecular weight of 600,000 to 1,200,000 (Da); the dosage is from 30 to 50 (kGy); and the “HA having a lowered molecular weight” has an average molecular weight of 1,700 to 2,500 (Da);
the production process wherein the “HA fraction” to which the electron beam is irradiated has an average molecular weight of 600,000 to 1,200,000 (Da); the dosage is from 50 to 80 (kGy); and the “HA having a lowered molecular weight” has an average molecular weight of 1,300 to 1,700 (Da).

Also, as a preferred embodiment of the molecular weight-lowering method of the present invention, the present invention provides a method for lowering the molecular weight of HA, which comprises irradiating an electron beam to an HA fraction (hereinafter referred to as “molecular weight-lowering method 2 of the present invention”).

Also, as a preferred embodiment of the molecular weight-lowering method 2 of the present invention, the present invention provides a method for lowering the molecular weight of HA, which comprises irradiating an electron beam to “an HA fraction which has an average molecular weight of 5,000 to 3,000,000 (Da) and is in a solid state” at a dosage of 5 to 400 (kGy). In addition, the following preferred embodiments of this molecular weight-lowering method are provided:
the method wherein the “HA fraction” to which the electron beam is irradiated has an average molecular weight of 200,000 to 3,000,000 (Da); the dosage is from 5 to 15 (kGy); and the HA after lowering the molecular weight has an average molecular weight of 100,000 to 200,000 (Da);
the method wherein the “HA fraction” to which the electron beam is irradiated has an average molecular weight of 100,000 to 3,000,000 (Da); the dosage is from 15 to 30 (kGy); and the HA after lowering the molecular weight has an average molecular weight of 60,000 to 100,000 (Da);
the method wherein the “HA fraction” to which the electron beam is irradiated has an average molecular weight of 50,000 to 3,000,000 (Da); the dosage is from 30 to 50 (kGy); and the HA after lowering the molecular weight has an average molecular weight of 30,000 to 60,000 (Da);
the method wherein the “HA fraction” to which the electron beam is irradiated has an average molecular weight of 30,000 to 3,000,000 (Da); the dosage is from 50 to 150 (kGy); and the HA after lowering the molecular weight has an average molecular weight of 20,000 to 30,000 (Da);
the method wherein the “HA fraction” to which the electron beam is irradiated has average molecular weight of 20,000 to 3,000,000 (Da); the dosage is from 150 to 250 (kGy); and the HA after lowering the molecular weight has an average molecular weight of 10,000 to 20,000 (Da);
the method wherein the “HA fraction” to which the electron beam is irradiated has an average molecular weight of 5,000 to 3,000,000 (Da); the dosage is from 250 to 350 (kGy); and the HA after lowering the molecular weight has an average molecular weight of 3,000 to 10,000 (Da).

Also, as another preferred embodiment of the molecular weight-lowering method 2 of the present invention, the present invention provides a method for lowering the molecular weight of HA, which comprises irradiating an electron beam to “an HA fraction which has an average molecular weight of 600,000 to 1,200,000 (Da) and is in a liquid state” at a dosage of 10 to 80 (kGy). In addition, the following preferred embodiments of this molecular weight-lowering method are provided:
the method wherein the “HA fraction” to which the electron beam is irradiated has an average molecular weight of 600,000 to 1,200,000 (Da); the dosage is from 10 to 30 (kGy); and the HA after lowering the molecular weight has an average molecular weight of 2,500 to 4,000 (Da);
the method wherein the “HA fraction” to which the electron beam is irradiated has an average molecular weight of 600,000 to 1,200,000 (Da); the dosage is from 30 to 50 (kGy); and the HA after lowering the molecular weight has an average molecular weight of 1,700 to 2,500 (Da);
the method wherein the “HA fraction” to which the electron beam is irradiated has an average molecular weight of 600,000 to 1,200,000 (Da); the dosage is from 50 to 80 (kGy); and the HA after lowering the molecular weight has an average molecular weight of 1,300 to 1,700 (Da).

Embodiments of the present invention are described as follows.

Production Process of the Present Invention

The production process of the present invention is a process for producing a saccharide having a lowered molecular weight, which comprises at least a step of irradiating an electron beam to a polysaccharide fraction.

The kinds, the molecular weight and the like of the “polysaccharide fraction” to which the electron beam is irradiated are not particularly limited, but it is preferably a GAG fraction. Examples of this “GAG fraction” include a fraction containing at least one species of GAGs selected from the group consisting of HA, CS (including CS-A, CS-C, CS-D, CS-E and the like), DS, KS, HS and Hep. Among these, an HA fraction is preferred.

An electron beam can be irradiated by using a general electron beam irradiation apparatus or the like. Although irradiation conditions and the like are not particularly limited too, the following conditions can be exemplified.

Electron Beam Irradiation Apparatus:

Dynamitron 5 MeV electron accelerator (manufactured by RDI)

| | |
|---|---|
| Voltage: | 2.0 MeV |
| Current: | 20.0 mA |
| Cart speed (periphery): | 18.0 m/minute |
| Irradiation direction: | one side |

The polysaccharide fraction may be put into a container capable of permeating electron beam, such as a plastic, glass, or metal, and be irradiated with an electron beam via this container, or it may be directly irradiated with an electron beam without using such a container. In addition, the polysaccharide fraction to be irradiated with an electron beam may be in a solid state (dried state) or a liquid state.

When the electron beam is irradiated to a polysaccharide fraction in this manner, a “saccharide having a lowered molecular weight” is produced due to lowering of the molecular weight of the polysaccharide. When the dosage of the irradiating electron beam is increased, a saccharide having a lower average molecular weight can be produced. Although the dosage of the irradiating electron beam can be optionally decided in response to the average molecular weight and the like of the desired “saccharide having a lowered molecular weight”, when the “polysaccharide fraction” (raw material) to be irradiated with an electron beam has an average molecular weight of 5,000 to 70,000 (Da) and is in a solid state, the “saccharide having a lowered molecular weight” having a desired average molecular weight n (Da) can be produced by calculating a dosage of d (kGy) which satisfies the following equation, and irradiating the dosage:
n=Me^{ad}

In this equation, “M” is an average molecular weight (Da) of the “polysaccharide fraction” (raw material) to be irradiated with an electron beam, and its numerical value can be set within the range of 5,000 to 70,000 (Da) according to the raw material. Also, “n” is an average molecular weight (Da) of the “saccharide having a lowered molecular weight”. Accordingly, “n” can be set to a numerical value (optional positive number) according to the average molecular weight of the “saccharide having a lowered molecular weight” to be produced. Also, “e” is the base of natural logarithm, and “a” is a number of −0.008 to −0.004.

This “a” is preferably a number of −0.008 to −0.005, more preferably a number of −0.0075 to −0.0050.

By using this equation, a proper irradiation dosage can be easily calculated from the average molecular weight of the “polysaccharide fraction” to be used as the production raw material (within the range of 5,000 to 70,000 (Da)) and the average molecular weight of the desired “saccharide having a lowered molecular weight”.

In addition, when the “polysaccharide fraction” (raw material) to be irradiated with an electron beam “has an average molecular weight of 5,000 to 3,000,000 (Da) and is an HA fraction in a solid state”, the “saccharide having a lowered molecular weight” having a desired average molecular weight can be produced by irradiating an electron beam at a dosage of 5 to 400 (kGy).

That is, when the desired average molecular weight of the “HA having a lowered molecular weight” is from 100,000 to 200,000 (Da), an “HA fraction” having an average molecular weight of 200,000 to 3,000,000 (Da) is employed as the raw material and an electron beam is irradiated thereto at a dosage of 5 to 15 (kGy).

In the same manner, when the desired average molecular weight of the “HA having a lowered molecular weight” is from 60,000 to 100,000 (Da), an “HA fraction” having an average molecular weight of 100,000 to 3,000,000 (Da) is employed as the raw material and an electron beam is irradiated thereto at a dosage of 15 to 30 (kGy).

In the same manner, when the desired average molecular weight of the “HA having a lowered molecular weight” is from 30,000 to 60,000 (Da), an “HA fraction” having an average molecular weight of 50,000 to 3,000,000 (Da) is employed as the raw material and an electron beam is irradiated thereto at a dosage of 30 to 50 (kGy).

In the same manner, when the desired average molecular weight of the “HA having a lowered molecular weight” is from 20,000 to 30,000 (Da), an “HA fraction” having an average molecular weight of 30,000 to 3,000,000 (Da) is employed as the raw material and an electron beam is irradiated thereto at a dosage of 50 to 150 (kGy).

In the same manner, when the desired average molecular weight of the “HA having a lowered molecular weight” is from 10,000 to 20,000 (Da), an “HA fraction” having an average molecular weight of 20,000 to 3,000,000 (Da) is employed as the raw material and the electron beam is irradiated thereto at a dosage of 150 to 250 (kGy).

In the same manner, when the desired average molecular weight of the “HA having a lowered molecular weight” is from 3,000 to 10,000 (Da), an “HA fraction” having an average molecular weight of 5,000 to 3,000,000 (Da) is employed as the raw material and the electron beam is irradiated thereto at a dosage of 250 to 350 (kGy).

Also, it is necessary as a matter of course that the average molecular weight of the “HA fraction” to be used as the raw material is larger than the average molecular weight of the “HA having a lowered molecular weight” to be produced, and the numerical value can be optionally set within the above-described range under such conditions.

The greatest characteristic of this production process is that the average molecular weight of the HA fraction to be used as the raw material can be set within a markedly broad range, and by irradiating a predetermined dosage of an electron beam thereto, an “HA fraction having a lowered molecular weight” having a predetermined average molecular weight can be produced, independent of the average molecular weight of the raw material.

In addition, when the “polysaccharide fraction” (raw material) to be irradiated with an electron beam “has an average molecular weight of 600,000 to 1,200,000 (Da) and is an HA fraction in a liquid state”, the “HA having a lowered molecular weight” which has a desired average molecular weight can be produced by irradiating an electron beam at a dosage of 10 to 80 (kGy). When an HA fraction of a liquid state is used as the raw material, the molecular weight can easily be lowered at a lower dosage than the case of using an HA fraction of a solid state.

That is, when the desired average molecular weight of the “HA having a lowered molecular weight” is from 2,500 to 4,000 (Da), an “HA fraction” having an average molecular weight of 600,000 to 1,200,000 (Da) is employed as the raw material and an electron beam is irradiated thereto at a dosage of 10 to 30 (kGy).

In the same manner, when the desired average molecular weight of the “HA having a lowered molecular weight” is from 1,700 to 2,500 (Da), an “HA fraction” having an average molecular weight of 600,000 to 1,200,000 (Da) is employed as the raw material and an electron beam is irradiated thereto at a dosage of 30 to 50 (kGy).

In the same manner, when the desired average molecular weight of the “HA having a lowered molecular weight” is from 1,300 to 1,700 (Da), an “HA fraction” having an average molecular weight of 600,000 to 1,200,000 (Da) is employed as the raw material and an electron beam is irradiated thereto at a dosage of 50 to 80 (kGy).

Also, the production process of the present invention may further contain other step (e.g., fractionation and purification steps and the like), so long as it contains at least a “step of irradiating an electron beam to a polysaccharide fraction”.

Molecular Weight-Lowering Method of the Present Invention

The molecular weight-lowering method of the present invention is a variation of the production process of the present invention modified based on the conception of the “molecular weight-lowering method”. Accordingly, its detailed description is the same as the above-described production process of the present invention, and the molecular weight-lowering method of the present invention can be easily understood by changing the “production process” of the production process of the present invention to “molecular weight-lowering method”.

The production process of the present invention and the molecular weight-lowering method of the present invention are markedly useful, because a saccharide having a lowered molecular weight, which is useful as a raw material of a medicament, a food and the like, can be produced conveniently, quickly, inexpensively and in a large amount. In addition, since sterilization can be carried out simultaneously with the lowering of the molecular weight by the electron beam irradiation, they are markedly useful in producing a sterilized saccharide having a lowered molecular weight conveniently, quickly and inexpensively in a large amount.

The present invention is described below more in detail based on Examples, but the present invention is not limited thereto.

The raw materials, electron beam irradiation method and the like used in Examples are as follows.

Raw Materials:

CS-A (bovine trachea origin; manufactured by Seikagaku Corporation)
CS-C (shark cartilage origin; manufactured by Seikagaku Corporation)
CS-D (shark cartilage origin; manufactured by Seikagaku Corporation)
CS-E (squid cartilage origin; manufactured by Seikagaku Corporation)
DS (cockscomb origin; manufactured by Seikagaku Corporation)
KS (shark cartilage origin; manufactured by Seikagaku Corporation)
HA (cockscomb origin; manufactured by Seikagaku Corporation)
   Electron Beam Irradiation Method and the Like:

The method was carried out by consigning to Japan Electron Beam Irradiation Service, Tsukuba Center (Midorigahara 4-16, Tsukuba, Ibaraki, Japan). An electron beam was irradiated by using an electron beam irradiation apparatus (Dynamitron 5 MeV electron accelerator; manufactured by RDI). Each material fraction was put into a plastic dish for preparation (when the raw material fraction is in the solid state) or a glass ampoule (when the raw material fraction is in the liquid state) and arranged on a cart for irradiation, and the electron beam was irradiated under the following conditions.

| | |
|---|---|
| Voltage: | 2.0 MeV |
| Current: | 20.0 mA |
| Cart speed (periphery): | 18.0 m/minute |
| Irradiation direction: | one side |

Whether or not a predetermined dosage was irradiated was confirmed by using a dosage measuring apparatus (U-2000 spectrophotometer; manufactured by Hitachi) and a dosimeter (CTA dosimeter FTR-125; manufactured by Fuji Photo Film).

Molecular Weight Analyzing Method:

The molecular weight was analyzed by gel permeation chromatography (GPC) under the following conditions.
Column: G6000PWXL (manufactured by Tosoh) (when a molecular weight of 50,000 or more is analyzed);
   G4000WXL+G3000PWXL (manufactured by Tosoh) (when a molecular weight of 50,000 or less is analyzed)
Solvent: 0.2 M NaCl
Flow rate: 0.5 ml/min
Detection: UV 205 nm
   Analysis of Oligosaccharide Composition:

An oligosaccharide composition was analyzed by HPLC under the following conditions. The elution position of the each saccharide was compared with those of standard substances (HA tetrasaccharide, hexasaccharide, octasaccharide and decasaccharide), and the concentration of each saccharide was roughly measured.
Column: YMCgel-PA120-S5 (manufactured by YMC)
Flow rate: 0.5 ml/min
Elution: density gradient by 40 mM to 200 mM and 60 mM to 200 mM sodium sulfate
Labeling: post-labeling with 2-cyanoacetamide
Detection: fluorescence detector Ex; 331 nm, Em; 383 nm
   Disaccharide composition analysis (analysis of the number, binding position and the like of sulfate group contained in disaccharide unit):
   (1) When CS or DS was Used

After 100 μl of each sample solution (about 200 μg/ml) was collected and adjusted to a total volume of 200 μl by adding 40 μl of 100 mM Tris-HCl buffer (pH 8.0) and chondroitinase ABC (83 mU; manufactured by Seikagaku Corporation), the reaction was carried out at 37° C. for 2 hours. The reaction solution was filtered through an ultrafiltration filter with nominal molecular weight cutoff of 10,000. The disaccharide composition was analyzed by subjecting the resulting filtrate to HPLC (column: YMC gel PA-120; manufactured by YMC).

(2) When KS was Used

After 100 μl of each sample solution (about 100 μg/ml) was collected and adjusted to a total volume of 200 μl by adding 40 μl of 100 mM sodium acetate buffer (pH 6.0) and keratanase II (1 mU; manufactured by Seikagaku Corporation), the reaction was carried out at 37° C. for 3 hours. The reaction solution was filtered through an ultrafiltration filter with nominal molecular weight cutoff of 10,000. The disaccharide composition was analyzed by subjecting the resulting filtrate to HPLC (column: YMC gel PA-120; manufactured by YMC).

### EXAMPLE 1

An average molecular weight was analyzed by irradiating various dosages of electron beams to HA fractions having various average molecular weights (in the solid state). The results are shown below.

Test 1: HA

| | |
|---|---|
| Before irradiation (0 kGy) | 2,500,000 (average molecular weight) |
| After 10 kGy irradiation | 199,000 (average molecular weight) |
| After 20 kGy irradiation | 99,000 (average molecular weight) |
| After 40 kGy irradiation | 52,000 (average molecular weight) |

Test 2: HA

| | |
|---|---|
| Before irradiation (0 kGy) | 1,500,000 (average molecular weight) |
| After 10 kGy irradiation | 134,000 (average molecular weight) |
| After 20 kGy irradiation | 74,000 (average molecular weight) |
| After 40 kGy irradiation | 43,000 (average molecular weight) |

Test 3: HA

| | |
|---|---|
| Before irradiation (0 kGy) | 1,100,000 (average molecular weight) |
| After 100 kGy irradiation | 26,500 (average molecular weight) |
| After 200 kGy irradiation | 15,000 (average molecular weight) |
| After 300 kGy irradiation | 6,400 (average molecular weight) |

Test 4: HA

| | |
|---|---|
| Before irradiation (0 kGy) | 493,000 (average molecular weight) |
| After 100 kGy irradiation | 26,500 (average molecular weight) |
| After 200 kGy irradiation | 16,500 (average molecular weight) |
| After 300 kGy irradiation | 6,500 (average molecular weight) |

Test 5: HA

| | |
|---|---|
| Before irradiation (0 kGy) | 298,700 (average molecular weight) |
| After 100 kGy irradiation | 26,200 (average molecular weight) |
| After 200 kGy irradiation | 13,000 (average molecular weight) |
| After 300 kGy irradiation | 7,200 (average molecular weight) |

Test 6: HA

| | |
|---|---|
| Before irradiation (0 kGy) | 62,000 (average molecular weight) |
| After 100 kGy irradiation | 23,000 (average molecular weight) |
| After 200 kGy irradiation | 15,500 (average molecular weight) |
| After 300 kGy irradiation | 7,000 (average molecular weight) |

Test 7: HA

| | |
|---|---|
| Before irradiation (0 kGy) | 6,100 (average molecular weight) |
| After 300 kGy irradiation | 4,200 (average molecular weight) |

Based on the above results, it was shown that the molecular weight is lowered to an almost constant average molecular weight when a certain level of a dosage is irradiated, independent of the average molecular weight of the HA to be used as the raw material.

### EXAMPLE 2

An average molecular weight was analyzed by irradiating various dosages of electron beams to HA fractions having various average molecular weights (aqueous solution; 10 mg/ml in concentration). The results are shown below.

| | |
|---|---|
| Before irradiation (0 kGy) | 850,000 (average molecular weight) |
| After 20 kGy irradiation | 3,200 (average molecular weight) |
| After 40 kGy irradiation | 1,900 (average molecular weight) |
| After 60 kGy irradiation | 1,500 (average molecular weight) |

Based on the above results, it was shown that the molecular weight can be lowered by electron beam even when the HA fraction to be used as the raw material is in a liquid state, and that the molecular weight is apt to be lowered in comparison with the case of solid state.

### EXAMPLE 3

The oligosaccharide composition in the fractions after electron beam irradiation obtained in Test 3, Test 4, Test 6 and Test 7 in Example 1 and in Example 2 was analyzed. The results are shown in Table 1.

TABLE 1

| | | Average | Concentration (%) | | | |
|---|---|---|---|---|---|---|
| | Dosage | molecular | Tetra- | Hexa- | Octa- | Deca- |
| Sample | (kGy) | weight (Da) | saccharide | saccharide | saccharide | saccharide |
| HA (Ex. 1, Test 3) | 100 | 26500 | 0.4 | 0.3 | 0.2 | 0.2 |
| | 200 | 15000 | 1.0 | 0.8 | 0.6 | 0.4 |
| | 300 | 6400 | 2.6 | 1.6 | 1.0 | 0.7 |
| HA (Ex. 1, Test 4) | 100 | 26500 | 0.3 | 0.2 | 0.2 | 0.1 |
| | 200 | 16500 | 0.9 | 0.8 | 0.6 | 0.4 |
| | 300 | 6500 | 2.1 | 1.6 | 1.0 | 1.0 |
| HA (Ex. 1, Test 6) | 100 | 23000 | 0.4 | 0.3 | 0.2 | 0.2 |
| | 200 | 15500 | 1.1 | 0.8 | 0.6 | 0.5 |
| | 300 | 7000 | 2.1 | 1.5 | 0.9 | 0.8 |
| HA (Ex. 1, Test 7) | 300 | 4200 | 5.3 | 4.5 | 4.0 | 3.2 |
| HA (Ex. 2) | 20 | 3200 | 2.0 | 2.2 | 2.2 | 1.1 |
| | 40 | 1900 | 4.5 | 4.0 | 3.2 | 1.6 |
| | 60 | 1500 | 6.4 | 5.8 | 3.4 | 1.6 |

It was confirmed from Table 1 that oligosaccharides having various sizes can be produced by electron beam irradiation. Accordingly, it was shown that the production process of the present invention can be used also as a “production process of oligosaccharide”.

### EXAMPLE 4

A disaccharide composition and an average molecular weight were analyzed by irradiating an electron beam of various dosages to various sulfated GAG fractions (solid state). The results are shown in Table 2 (a case of using CS or DS) and Table 3 (a case of using KS). In this connection, the terms “0S”, “6S”, “4S”, “SD”, “SB”, “SE”, “M-KS” and “D-KS” used in the tables are abbreviations respectively showing specific disaccharide structures including the number, binding position and the like of sulfate groups.

TABLE 2

| | | | | | | | | Average |
|---|---|---|---|---|---|---|---|---|
| Sam- | Dosage | Disaccharide composition ratio (%) | | | | | | molecular |
| ple | (kGy) | 0S | 6S | 4S | SD | SB | SE | weight (Da) |
| CS-D | 0 | 0.8 | 44.5 | 27.9 | 23.0 | — | 3.8 | 28,826 |
| | 100 | 1.2 | 46.8 | 26.5 | 21.9 | — | 3.6 | 15,577 |
| | 200 | 1.9 | 46.9 | 25.8 | 22.0 | — | 3.5 | 8,651 |
| | 300 | 2.4 | 47.3 | 25.6 | 21.3 | — | 3.4 | 4,409 |
| CS-C | 0 | 5.5 | 45.8 | 32.8 | 13.8 | — | 2.1 | 8,336 |
| | 100 | 5.7 | 46.2 | 32.7 | 13.4 | — | 2.0 | 4,451 |
| | 200 | 6.2 | 46.1 | 32.5 | 13.1 | — | 2.0 | 3,871 |
| | 300 | 6.4 | 46.4 | 31.9 | 13.2 | — | 2.0 | 3,013 |
| CS-A | 0 | 5.0 | 33.4 | 60.6 | 0.5 | — | 0.4 | 14,696 |
| | 100 | 5.4 | 33.4 | 60.2 | 0.5 | — | 0.4 | 11,222 |
| | 200 | 6.0 | 33.6 | 59.6 | 0.4 | — | 0.4 | 8,555 |
| | 300 | 6.5 | 33.7 | 59.0 | 0.4 | — | 0.4 | 4,834 |
| CS-E | 0 | 4.0 | 14.1 | 20.2 | — | — | 61.7 | 42,316 |
| | 100 | 3.8 | 14.1 | 20.2 | — | — | 61.9 | 10,170 |
| | 200 | 3.8 | 14.4 | 21.0 | — | — | 60.9 | 7,756 |
| | 300 | 4.3 | 14.6 | 21.0 | — | — | 60.1 | 4,465 |
| DS | 0 | 3.9 | 7.3 | 82.2 | 1.1 | 5.5 | — | 45,942 |
| | 100 | 4.0 | 7.2 | 82.2 | 1.1 | 5.7 | — | 21,084 |
| | 200 | 3.9 | 7.2 | 82.4 | 1.0 | 5.5 | — | 11,523 |
| | 300 | 4.5 | 7.2 | 81.8 | 1.0 | 5.5 | — | 4,974 |

TABLE 3

| | | |
|---|---|---|
| | Disaccharide | |
| | composition | Average |
| Dosage | ratio (%) | molecular |

| Sample | (kGy) | M-KS | D-KS | weight (Da) |
|---|---|---|---|---|
| KS | 0 | 1.2 | 98.8 | 12,167 |
| | 100 | 1.5 | 98.5 | 9,959 |
| | 200 | 1.7 | 98.3 | 6,739 |
| | 300 | 2.0 | 98.0 | 5,283 |
| KS | 0 | 2.0 | 98.0 | 7,373 |
| | 100 | 3.1 | 96.9 | 4,743 |
| | 200 | 2.9 | 97.1 | 4,974 |
| | 300 | 3.0 | 97.0 | 4,451 |

Also, the term “M-KS” means that the sulfate group is kept only on the 6-position of the N-acetylglucosamine (GlcNAc) residue in the disaccharide unit of KS, and the term “D-KS” means that the sulfate group is kept on both of the 6-position of the galactose (Gal) residue and the 6-position of N-acetylglucosamine (GlcNAc) residue in the disaccharide unit of KS.

Based on the results, it was shown that, similar to the case of HA fractions, the molecular weight can be lowered by an electron beam even when the raw material is sulfated GAG fractions, and that the electron beam irradiation hardly has influence upon the disacharide composition (the number, binding position and the like of sulfate groups contained in the disaccharide unit).

Also, as a result of regression analysis carried out by using the results of Example 1 and Example 4, it was found that the following relational expression is formed when the average molecular weight (M) of a polysaccharide fraction to be used as the raw material is from 5,000 to 70,000.
n=Me^{ad}
M: Average molecular weight (Da) of the polysaccharide fraction to be used as the raw material (from 5,000 to 70,000)
n: Average molecular weight (Da) after electron beam irradiation
e: Base of natural logarithm
a: A number of −0.0075 to −0.0050
d: Dosage (kGy) of irradiated electron beam

In addition, when nuclear magnetic resonance spectrum (NMR) analysis was carried out on the fractions after electron beam irradiation, it was suggested that irradiation of electron beam does not have fatal influence upon the skeletal structure of polysaccharide.

While the present invention has been described in detail and with reference to specific embodiments thereof, it will be apparent to one of skill in the art that various changes and modifications can be made therein without departing from the spirit and scope thereof. All references cited herein are incorporated in their entirety.

1. A process for producing a saccharide having a lowered molecular weight, which comprises at least a step of irradiating an electron beam to a polysaccharide fraction.

2. The process according to claim 1, wherein the polysaccharide fraction to which the electron beam is irradiated is in a solid state, and the electron beam is irradiated at a dosage of d (kGy) which satisfies the following equation:
n=Me^{ad}
wherein M represents a weight average molecular weight (Da) of the polysaccharide fraction and is a number of 5,000 to 70,000; n represents a weight average molecular weight (Da) of the saccharide having a lowered molecular weight and is an optional positive number; e is the base of natural logarithm; and a is a number of −0.008 to −0.004.

3. The process according to claim 2, wherein a is a number of −0.008 to −0.005.

4. The process according to claim 3, wherein a is a number of −0.0075 to −0.0050.

5. The process according to claim 1, wherein the polysaccharide fraction to which the electron beam is irradiated is a glycosaminoglycan fraction.

6. The process according to claim 5, wherein the glycosaminoglycan fraction is a fraction comprising at least one species of glycosaminoglycans selected from the group consisting of hyaluronic acid, chondroitin sulfate, dermatan sulfate, keratan sulfate, heparan sulfate and heparin.

7. A method for lowering the molecular weight of a polysaccharide, which comprises irradiating an electron beam to a polysaccharide fraction.

8. The method according to claim 7, wherein the polysaccharide fraction to which the electron beam is irradiated is in a solid state, and the electron beam is irradiated at a dosage of d (kGy) which satisfies the following equation:
n=Me^{ad}
wherein M represents a weight average molecular weight (Da) of the polysaccharide fraction and is a number of 5,000 to 70,000; n represents a weight average molecular weight (Da) of the saccharide having a lowered molecular weight and is an optional positive number; e is the base of natural logarithm; and a is a number of −0.008 to −0.004.

9. The method according to claim 8, wherein a is a number of −0.008 to −0.005.

10. The method according to claim 9, wherein a is a number of −0.0075 to −0.0050.

11. The method according to claim 7, wherein the polysaccharide fraction to which the electron beam is irradiated is a glycosaminoglycan fraction.

12. The method according to claim 11, wherein the glycosaminoglycan fraction is a fraction containing at least one species of glycosaminoglycans selected from the group consisting of hyaluronic acid, chondroitin sulfate, dermatan sulfate, keratan sulfate, heparan sulfate and heparin.

13. A process for producing hyaluronic acid having a lowered molecular weight, which comprises at least a step of irradiating an electron beam to a hyaluronic acid fraction.

14. The process according to claim 13, wherein the hyaluronic acid fraction to which the electron beam is irradiated has a weight average molecular weight of 5,000 to 3,000,000 (Da) and is in a solid state, and the electron beam is irradiated at a dosage of 5 to 400 (kGy).

15. The process according to claim 14, wherein the hyaluronic acid fraction to which the electron beam is irradiated has a weight average molecular weight of 200,000 to 3,000,000 (Da); the dosage is from 5 to 15 (kGy); and the hyaluronic acid having a lowered molecular weight has a weight average molecular weight of 100,000 to 200,000 (Da).

16. The process according to claim 14, wherein the hyaluronic acid fraction to which the electron beam is irradiated has a weight average molecular weight of 100,000 to 3,000,000 (Da); the dosage is from 15 to 30 (kGy); and the hyaluronic acid having a lowered molecular weight has a weight average molecular weight of 60,000 to 100,000 (Da).

17. The process according to claim 14, wherein the hyaluronic acid fraction to which the electron beam is irradiated has a weight average molecular weight of 50,000 to 3,000,000 (Da); the dosage is from 30 to 50 (kGy); and the hyaluronic acid having a lowered molecular weight has a weight average molecular weight of 30,000 to 60,000 (Da).

18. The process according to claim 14, wherein the hyaluronic acid fraction to which the electron beam is irradiated has a weight average molecular weight of 30,000 to 3,000,000 (Da); the dosage is from 50 to 150 (kGy); and the hyaluronic acid having a lowered molecular weight has a weight average molecular weight of 20,000 to 30,000 (Da).

19. The process according to claim 14, wherein the hyaluronic acid fraction to which the electron beam is irradiated has a weight average molecular weight of 20,000 to 3,000,000 (Da); the dosage is from 150 to 250 (kGy); and the hyaluronic acid having a lowered molecular weight has a weight average molecular weight of 10,000 to 20,000 (Da).

20. The process according to claim 14, wherein the hyaluronic acid fraction to which the electron beam is irradiated has a weight average molecular weight of 5,000 to 3,000,000 (Da); the dosage is from 250 to 350 (kGy); and the hyaluronic acid having a lowered molecular weight has a weight average molecular weight of 3,000 to 10,000 (Da).

21. A process for producing hyaluronic acid having a lowered molecular weight, which comprises at least a step of irradiating an electron beam to a hyaluronic acid fraction which has a weight average molecular weight of 600,000 to 1,200,000 (Da) and is in a liquid state at a dosage of 10 to 80 (kGy).

22. The process according to claim 21, wherein the hyaluronic acid fraction to which the electron beam is irradiated has a weight average molecular weight of 600,000 to 1,200,000 (Da); the dosage is from 10 to 30 (kGy); and the hyaluronic acid having a lowered molecular weight has a weight average molecular weight of 2,500 to 4,000 (Da).

23. The process according to claim 21, wherein the hyaluronic acid fraction to which the electron beam is irradiated has a weight average molecular weight of 600,000 to 1,200,000 (Da); the dosage is from 30 to 50 (kGy); and the hyaluronic acid having a lowered molecular weight has a weight average molecular weight of 1,700 to 2,500 (Da).

24. The process according to claim 21, wherein the hyaluronic acid fraction to which the electron beam is irradiated has a weight average molecular weight of 600,000 to 1,200,000 (Da); the dosage is from 50 to 80 (kGy); and the hyaluronic acid having a lowered molecular weight has a weight average molecular weight of 1,300 to 1,700 (Da).

25. A method for lowering the molecular weight of hyaluronic acid, which comprises irradiating an electron beam to a hyaluronic acid fraction.

26. The method according to claim 25, wherein the hyaluronic acid fraction to which the electron beam is irradiated has a weight average molecular weight of 5,000 to 3,000,000 (Da) and is in a solid state; and the electron beam is irradiated at a dosage of 5 to 400 (kGy).

27. The method according to claim 26, wherein the hyaluronic acid fraction to which the electron beam is irradiated has a weight average molecular weight of 200,000 to 3,000,000 (Da); the dosage is from 5 to 15 (kGy); and the hyaluronic acid after lowering the molecular weight has a weight average molecular weight of 100,000 to 200,000 (Da).

28. The method according to claim 26, wherein the hyaluronic acid fraction to which the electron beam is irradiated has a weight average molecular weight of 100,000 to 3,000,000 (Da); the dosage is from 15 to 30 (kGy); and the hyaluronic acid after lowering the molecular weight has a weight average molecular weight of 60,000 to 100,000 (Da).

29. The method according to claim 26, wherein the hyaluronic acid fraction to which the electron beam is irradiated has a weight average molecular weight of 50,000 to 3,000,000 (Da); the dosage is from 30 to 50 (kGy); and the hyaluronic acid after lowering the molecular weight has a weight average molecular weight of 30,000 to 60,000 (Da).

30. The method according to claim 26, wherein the hyaluronic acid fraction to which the electron beam is irradiated has a weight average molecular weight of 30,000 to 3,000,000 (Da); the dosage is from 50 to 150 (kGy); and the hyaluronic acid after lowering the molecular weight has a weight average molecular weight of 20,000 to 30,000 (Da).

31. The method according to claim 26, wherein the hyaluronic acid fraction to which the electron beam is irradiated has a weight average molecular weight of 20,000 to 3,000,000 (Da); the dosage is from 150 to 250 (kGy); and the hyaluronic acid after lowering the molecular weight has a weight average molecular weight from 10,000 to 20,000 (Da).

32. The method according to claim 26, wherein the hyaluronic acid fraction to which the electron beam is irradiated has a weight average molecular weight of 5,000 to 3,000,000 (Da); the dosage is from 250 to 350 (kGy); and the hyaluronic acid after lowering the molecular weight has a weight average molecular weight of 3,000 to 10,000 (Da).

33. A method for lowering the molecular weight of hyaluronic acid, which comprises irradiating an electron beam to a hyaluronic acid fraction which has a weight average molecular weight of 600,000 to 1,200,000 (Da) and is in a liquid state at a dosage of 10 to 80 (kGy).

34. The method according to claim 33, wherein the hyaluronic acid fraction to which the electron beam is irradiated has a weight average molecular weight of 600,000 to 1,200,000 (Da); the dosage is from 10 to 30 (kGy); and the hyaluronic acid after lowering the molecular weight has a weight average molecular weight of 2,500 to 4,000 (Da).

35. The method according to claim 33, wherein the hyaluronic acid fraction to which the electron beam is irradiated has a weight average molecular weight of 600,000 to 1,200,000 (Da); the dosage is from 30 to 50 (kGy); and the hyaluronic acid after lowering the molecular weight has a weight average molecular weight of 1,700 to 2,500 (Da).

36. The method according to claim 33, wherein the hyaluronic acid fraction to which the electron beam is irradiated has a weight average molecular weight of 600,000 to 1,200,000 (Da); the dosage is from 50 to 80 (kGy); and the hyaluronic acid after lowering the molecular weight has a weight average molecular weight of 1,300 to 1,700 (Da).
